(19) United States
(12) Patent Application Publication
(10) Pub. No.: US 2019/0311706 A1
(43) Pub. Date: Oct. 10, 2019

(54) ADAPTIVE NOISE CANCELLATION FOR MOBILE RADIOGRAPHY SYSTEM

(71) Applicant: CARESTREAM HEALTH, INC., Rochester, NY (US)

(72) Inventor: Scott T. MACLAUGHLIN, Pittsford, NY (US)

(21) Appl. No.: 15/948,132

(22) Filed: Apr. 9, 2018

(51) Int. Cl. G10K 11/178; A61B 6/00

(52) U.S. Cl. CPC G10K 11/17823; A61B 6/4405; G10K 2210/3028; G10K 2210/116; G10K 2210/3044; G10K 11/17854

(57) ABSTRACT

A mobile x-ray system having an x-ray source and an electronic control system for firing of the x-ray source includes a sound cancellation system. A microphone is used to detect incoming sound waves and a speaker is used to emit a canceling audio signal to defeat the detected incoming sound waves. The canceling audio signal destructively interferes with the incoming sound waves to attenuate or cancel at least portion of the detected incoming sound waves.

### FIELD OF THE INVENTION

The invention relates generally to the field of medical imaging and more particularly relates to systems and methods for adaptive noise cancellation in a mobile radiography system.

### BACKGROUND

Mobile radiography systems are of particular value in intensive care unit (ICU) and other environments where timely acquisition of a radiographic image is important. Because it can be wheeled around the ICU or other area and brought directly to the patient's bedside, a mobile radiography system allows an attending physician or clinician to have recent information on the condition of a patient and helps to reduce the risks entailed in moving patients to stationary equipment in the radiological facility.

The perspective view of FIG. 1 shows an example of a mobile radiography system 60 that can be employed for computed radiography (CR) and/or digital radiography (DR). A mobile radiography system 60 has a frame 62 that includes wheels 64 attached thereto and a display 61 for display of obtained images, a graphical user interface and related data, and a control panel 74 that allows selective operation of the mobile radiography system 60 and communicates with exposure components for firing of an x-ray source 68 as well as related functions such as storing, transmitting, modifying, and printing of the obtained radiographic images.

One or more DR detectors 70 can be carried within mobile radiography system 60 in a slot 69 configured to recharge the DR detector 70 when inserted therein. A central processing system 72 provides an electronic control system that executes logic functions for operating the mobile radiography system 60, including control over movement and positioning of an x-ray head 67 having an x-ray source 68, which may be provided on an adjustable column 66. The electronic control system provided by processing system 72 communicates with the x-ray head 67 for controlling actuation and firing of the x-ray source 68.

The mobile radiography system 60 has an internal battery 80 or other self-contained power source disposed within or coupled to frame 62 and used to power the various sub-systems of the mobile radiography system 60, including a transport drive system 76 with motors and other actuators and drive components that facilitate movement of the mobile radiography system 60 to different sites, one or more computers or dedicated logic processors that control various functions, displays that provide operator interface utilities and display imaging results, wireless transmitters and detectors, collapsible columns and other positioning facilitators, collimator lights, the x-ray source, and other functions. Typically, battery 80 is provided as a bank of multiple battery cells, such as lead-acid batteries. A handle 58 provides a steering device when rollably transporting the mobile radiography system 60.

One difficulty with mobile radiography system use in the clinical and hospital environment relates to system noise. While some measure of noise reduction can result from careful mechanical design and noise abatement practices, there is still some residual noise produced by the mobile radiography system, in transport, setup, and imaging phases of operation. Moreover, noise reduction and prevention techniques are often compromised and become less effective with equipment wear and degradation due to aging.

Ambient noise levels are acknowledged to be a chronic problem in the hospital environment. Noise resulting from motorized movement and operation of various types of test and diagnostic equipment and from operation of cleaning and maintenance equipment, noise from setup and construction, personnel noise, audio paging noises and alarms, elevator noise, noise from HVAC system operation, noise from televisions and other devices in patient rooms and waiting areas, and noise from other sources combine to drive ambient noise levels well above recommended guidelines. Some exemplary guidelines established for noise control from regulatory authorities include the following:
World Health Organization: 30 dB maximum in hospital rooms;
ANSI Standard 512.2-1995: range 25-40 dB depending on the type of room;
U.S. EPA: not to exceed 45 dB.

Unfortunately, actual hospital noise levels not only exceed current industry guidelines, but have also been steadily increasing as more equipment is introduced into the hospital setting. Thus, it can be readily appreciated that there would be benefits to methods and systems that help to reduce noise in hospital and clinical environments.

The discussion above is merely provided for general background information and is not intended to be used as an aid in determining the scope of the claimed subject matter.

### SUMMARY

An aspect of this application is to advance the art of medical digital radiography and to address, in whole or in part, at least the foregoing and other deficiencies of the related art. It is another aspect of this application to provide in whole or in part, at least the advantages described herein. For example, certain exemplary embodiments of the application address the need to reduce noise generated by the mobile radiography system. In addition, embodiments of the present disclosure also address the problem of reducing noise from nearby sources.

A mobile x-ray system having an x-ray source and an electronic control system for firing of the x-ray source includes a sound cancellation system. A microphone is used to detect incoming sound waves and a speaker is used to emit a canceling audio signal to defeat the detected incoming sound waves. The canceling audio signal destructively interferes with the incoming sound waves to attenuate or cancel at least portion of the detected incoming sound waves.

In one embodiment, a mobile digital radiography system includes an x-ray source controlled by an electronic control system. A user interface is provided for an operator to selectively operate the x-ray source. A sound cancellation system is configured to detect characteristics of incoming sound waves and to generate an outgoing audio interference signal in response thereto. The outgoing audio interference signal comprises destructive audio properties with respect to the incoming sound waves so that the outgoing audio interference signal cancels at least portions of the incoming sound waves.

In one embodiment, a method comprises providing a mobile radiography system having a wheeled transport frame which detects incoming sound waves and generates a cancelling audio signal having a predetermined phase shift relative to the detected incoming sound waves.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a perspective view of a mobile radiography system.

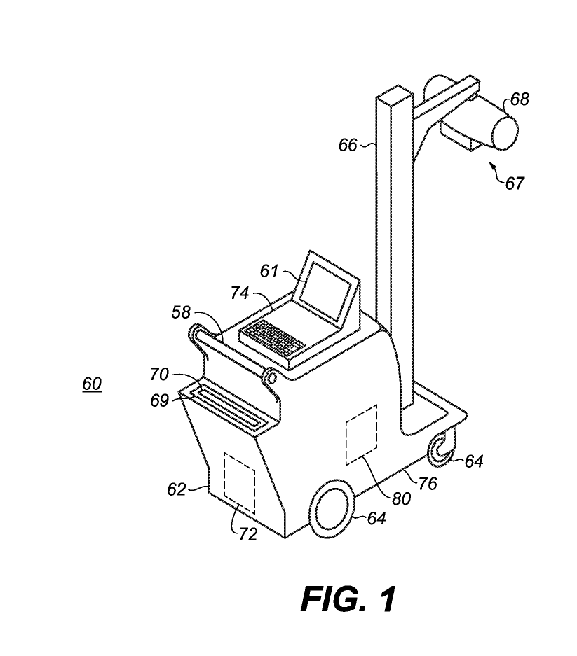

FIG. 2 is a wave form diagram that shows principles of constructive and destructive sound wave interference.

FIG. 3 is a schematic diagram that shows an adaptive noise cancellation system provided as a component of a mobile radiography system.

FIG. 4 is a perspective view of a medical treatment facility having multiple mobile radiography systems.

FIG. 5 is an operator interface for noise cancellation setup.

### DESCRIPTION OF EXEMPLARY EMBODIMENTS

In the context of the present disclosure, the phrase “in signal communication” indicates that two or more devices and/or components are capable of communicating with each other via signals that travel over some type of signal path. Signal communication may be wired or wireless. The signals may be digital communication, power, digital data, or energy signals. The signal paths may include physical, electrical, magnetic, electromagnetic, optical, wired, and/or wireless connections between the first device and/or component and second device and/or component. The signal paths may also include additional devices and/or components between the first device and/or component and the second device and/or component.

An embodiment of the present disclosure addresses the problem of noise or sound generated by the mobile radiography system as well as sound generated in the ambient environment near the mobile radiography system, such as equipment within the same room, by applying principles of adaptive noise cancellation (ANC). Adaptive Noise Cancellation utilizes sensing capabilities, such as a microphone and connected processor, and adaptive filter techniques to detect and/or record and/or characterize the ambient sounds and respond by generating a counter-signal, wherein the generated audio counter-signal effectively cancels the energy of the noise signal using destructive interference.

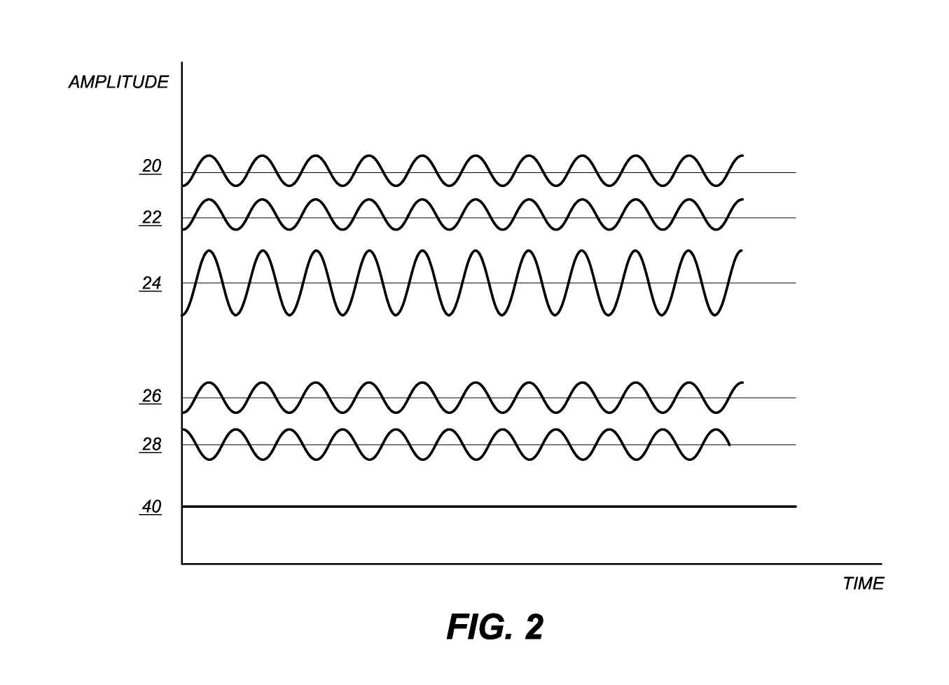

The wave form diagram of FIG. 2 illustrates how audio signal constructive and destructive interference operates for sound waves and other physical periodic signals, in general. Signals 20 and 22 are exemplary audio wave forms, or audio signals, having the same frequency—meaning that they have the same number of cycles per unit of time. Exemplary signals 20 and 22 are also in-phase, meaning that their maximum values (peaks) and minimum values (valleys) coincide in time, or occur simultaneously. The sum of in-phase exemplary audio signals 20 and 22 is represented as signal 24, which also has the same frequency and phase of the addend in-phase audio signals 20 and 22 and which is amplified as shown by the greater amplitude between the peaks and valleys of audio signal 24. The increased amplitude of signal 24 demonstrates constructive interference of in-phase audio signals 20 and 22.

Conversely, periodic audio signals 26 and 28 have the same frequency but are 180 degrees out of phase, meaning that the signal peaks (maximums) and valleys (minimums) of the two signals 26 and 28 are opposed, so that a maximum peak of one audio signal coincides in time with a minimum valley of the other audio signal. Signals 26 and 28 are exemplary audio signals having the same frequency. The sum of exemplary audio signals 26 and 28 is represented as signal 40 having an amplitude of zero. The zero amplitude of signal 40 demonstrates destructive interference of audio signals 26 and 28 in which the intensity, or sound energy, for the 180 degrees out-of-phase audio signals 26 and 28 is defeated by adding them. The two audio signals 26 and 28 cancel each other, so that the resulting sum of these audio signals, signal 40, is a null or zero audio signal.

In order to reduce or eliminate a sound signal, the approach used in ANC is to generate, under program control, and emit a corresponding counter-signal that emulates or copies at least some portion of undesired sound waves, i.e., noise, with components of the counter-signal having the same frequency as the detected noise, but with a phase shift of 180 degrees, as in the example of signals 26 and 28 of FIG. 2 described above. In one embodiment, ANC provides a programmed algorithm that echoes, records, or copies, at least a portion of the detected noise, applies a predetermined phase shift thereto and then emits the reconstructed programmed counter-signal audio, or sound having a predesigned destructive interference property.

In order to track and detect noise, ANC uses digital adaptive filters that are configured with a programmed impulse response or transfer function that allows real-time tracking of sound signals, allowing the ANC system to respond almost instantaneously with a destructively interfering (canceling) counter-signal, also termed an anti-noise signal, that can include audio components having the same frequency as the noise signal but with an appropriately shifted phase.

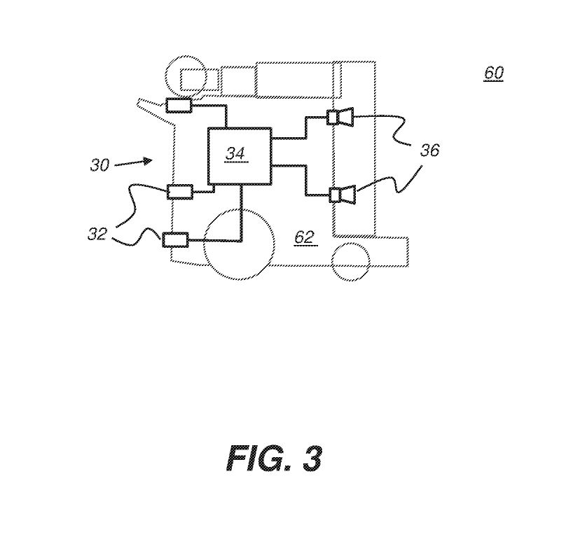

FIG. 3 is a schematic diagram of an adaptive noise cancellation system 30 provided as a component of mobile radiography system 60. Adaptive noise cancellation system 30 includes one or more microphones 32 disposed at suitable locations about mobile radiography system 60 in order to detect noise from mobile radiography system 60 itself and, alternately, to detect ambient noise from other sources in the hospital or clinical facility environment. A signal generator 34 is electrically connected to the microphones 32 to receive and process noise energy detected thereby, in order to generate the canceling audio counter-signal for output over one or more speakers 36 that are mounted at one or more locations about frame 62 of the mobile radiography system 60. The cancellation noise or audio counter-signal that is generated may be programmed to correspond to both the intensity level (dB) and frequency of the detected ambient noise. Signal generator 34 may include components such as one or more adaptive filters configured to track and record detected noise and to generate responsive audio signals that can be emitted over speakers to defeat or attenuate the detected ambient noise.

There are a number of algorithmic approaches to noise cancellation known to those skilled in the noise cancellation arts. Among methods familiar to those skilled in the noise cancellation arts are least-mean-squared (LMS) algorithms and their variants, such as the Filtered X algorithm, for example. In general, the LMS algorithm iteratively correlates an error or residual noise signal with a reference signal, then multiplies results by an adaptation rate constant used for adaptive filter adjustment. Repeated iterations of the algorithm converge on wave form values having properties that attenuate or cancel the average power (amplitude) in the ambient noise signal.

As described previously, the adaptive noise cancellation activity of mobile radiography system 60 can be configured to track and generate canceling anti-noise audio signals to defeat noise generated by mobile radiography system 60 as well as ambient noise in the environment near the mobile radiography system 60. This enables mobile radiography system 60 to serve an active role in noise abatement in the hospital or clinical environment, whether or not the mobile radiography system 60 is actively in use for radiography imaging.

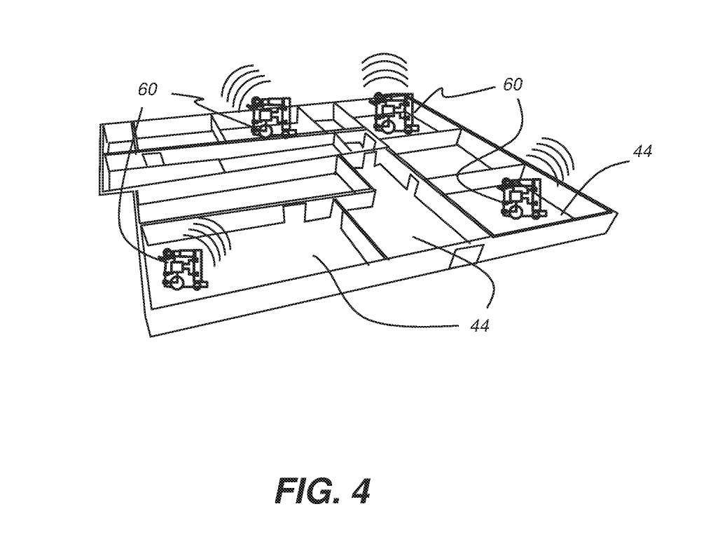

FIG. 4 is a perspective view of rooms 44 in a hospital ward or clinical setting having a number of mobile radiography systems 60 generating noise cancellation signals as described herein. Each mobile radiography system 60 generates noise cancellation signals within its local area, such as within a perimeter of 8 to 14 feet, for example. These mobile radiography systems 60 can be performing various functions related to normal use modes, such as being in a recharge mode, in a transport mode between patient rooms, in a set up mode for imaging, or actively acquiring an x-ray image, for example.

In this way, mobile radiography system 60 or other mobile diagnostic system that is suitably equipped with adaptive noise cancellation system 30 can serve a dual function, helping to curb excessive noise in hospital wards and other patient care areas. Because of its overall portability and its versatile application to numerous types of imaging applications, mobile radiography system 60 can be particularly useful for noise cancellation in a hospital or other patient care environment. This can include noise reduction capability used where it is particularly needed, such as in the intensive care unit (ICU), emergency room, neonatal intensive care unit (NICU), and at a patient bedside in various other environments.

The adaptive capability of noise cancellation system 30 can be particularly useful for mobile radiography systems used for patient diagnostics and treatment, including not only mobile radiography system 60, but other systems as well.

With complex mechanical systems of any type, noise characteristics can change over time due to wear, aging, and use. For wear components in particular, such as bearings, pulleys, brakes, motors, gears, and couplings, repeated use and aging can alter the characteristic frequencies and intensity of sound over time, so that squeaks, rattling, buzzing, and other mechanical sounds change. This factor makes it impractical to develop sophisticated noise cancellation systems that employ fixed sound profiles for a particular system, such as mobile radiography system 60. Adaptive systems, however, allow the noise cancellation features to respond dynamically and can correct for changing noise conditions of the equipment and its surrounding environment.

An embodiment of the present disclosure allows the operator to activate or de-activate the adaptive noise cancellation as needed, such as by providing a switch to turn off the noise canceling system. This feature can be used to facilitate communication between the technician and patient or other personnel that might otherwise be made difficult in the immediate locale of the mobile radiography system 60.

A measure of selectivity can be provided for “tuning” the response of the adaptive noise cancellation feature. This can include built-in adjustments, so that no attempt is made to provide a counter-signal for voice conversation, such as between patient and technician. Voice patterns have characteristic frequency distributions, or a frequency range that can be readily identified and distinguished from other noise sources, such as equipment noise, HVAC noise, and the like. Such characteristic frequency ranges may be selectively activated or deactivated in the noise canceling system described herein. Thus, the adaptive noise cancellation system 30 can be programmed to bypass or prevent noise cancellation in selected frequency ranges from alarms, personnel paging messages, human voice, and other urgent audio frequency ranges that are important to the operator and other personnel.

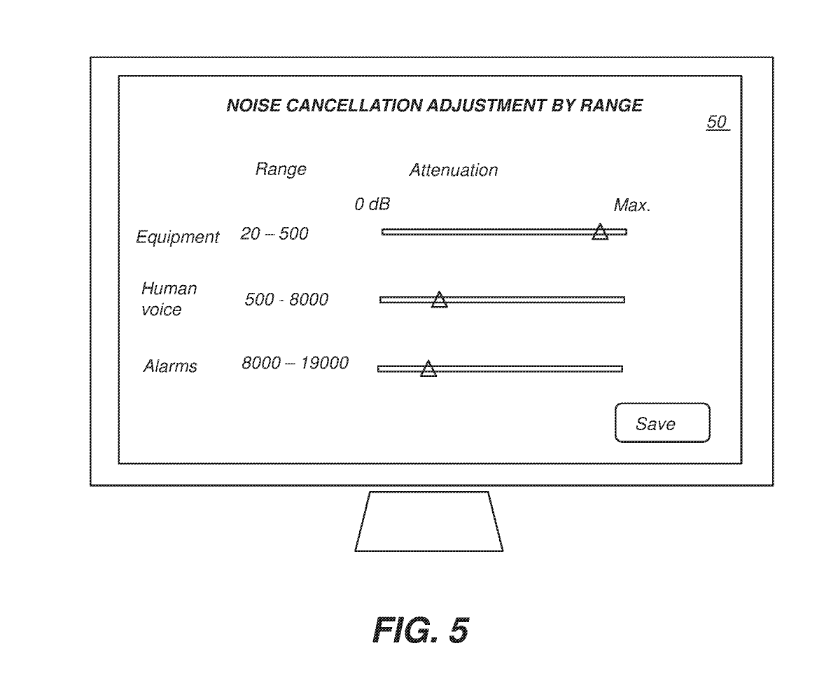

According to an embodiment of the present disclosure, ranges for noise cancellation can be adjusted by the operator/technician, to provide variable amounts of cancellation depending on the overall frequency pattern and noise source. The operator interface screen of FIG. 5 shows an interface 50 for noise cancellation programmability. Variable noise attenuation can be provided for different signal types, such as for voice, equipment, and alarm signals, for example. Variable adjustment for sensitivity of the response to various detected frequency levels can also be provided, as shown in FIG. 5. Thus, for example, only a low level of noise compensation can be provided for detected voice signals, with higher levels of noise abatement for system equipment or other equipment in the hospital or clinical environment.

According to an embodiment of the present disclosure, the noise cancellation system 30 can include learning software, such as software designed using neural network techniques, that enables the system to sense the various types of noise encountered in daily rounds and to distinguish between undesirable noise where noise cancellation is beneficial and useful sound that should not cause anti-noise signals to be generated.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing system to produce a machine, such that the instructions, which execute via the processor of the computer or other programmable data processing system, create means for implementing the functions/acts specified in the flowchart and/or block diagram block or blocks.

These computer program instructions may also be stored in a computer readable medium that can direct a computer, other programmable data processing system, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing system, or other devices to cause a series of operational steps to be performed on the computer, other programmable system or other devices to produce a computer implemented process such that the instructions which execute on the computer or other programmable system provide processes for implementing the functions/acts specified in the flowchart and/or block diagram block or blocks.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A mobile digital radiography system comprising:
an x-ray head comprising an x-ray source;
an electronic control system configured to communicate with the x-ray head for controlling firing of the x-ray source;
a control interface for an operator to selectively operate the electronic control system; and
a sound cancellation system including a microphone, a speaker, an interface screen, and a plurality of user selectable frequency ranges presented on the interface screen to a user of the mobile digital radiography system, the sound cancellation system configured to detect characteristics of incoming sound waves using the microphone and to generate an outgoing audio interference signal emitted over the speaker in the one or more of the frequency ranges selected by the user in response thereto,
wherein the outgoing audio interference signal comprises destructive audio properties in the one or more of the frequency ranges selected by the user with respect to the incoming sound waves, and wherein at least portions of the outgoing audio interference signal emitted over the speaker attenuate at least portions of the incoming sound waves in the one or more of the frequency ranges selected by the user using destructive interference.

2. The system of claim 1, wherein the sound cancellation system is configured to detect characteristics of incoming sound waves generated by the mobile radiography system and incoming sound waves generated by an ambient sound source.

3. The system of claim 1, wherein the electronic control system is further configured to selectively adjust a frequency of the outgoing audio interference signal according to a frequency of the incoming sound waves.

4. The system of claim 1, further comprising an operator control configured to turn off the sound cancellation system.

5. The system of claim 1, wherein the sound cancellation system is further configured to generate an outgoing audio interference signal having a predetermined phase shift with respect to the detected incoming sound waves.

6. The system of claim 5, wherein the sound cancellation system is further configured to copy the detected incoming sound waves and to generate the outgoing audio interference signal such that the outgoing audio interference signal is phase shifted with respect to the incoming sound waves.

7. The system of claim 1, wherein the sound cancellation system is further configured to detect the characteristics of the incoming sound waves using the microphone and to determine to not generate an outgoing audio interference signal in response to detecting certain preselected characteristics of the incoming sound waves.

8. A method comprising:
providing a mobile radiography system having a wheeled transport frame, a microphone, a speaker, and an interface screen, each electrically connected to a processor;
detecting incoming sound waves using the microphone and the processor electrically connected thereto;
presenting a plurality of frequency ranges on the interface screen for selection by an operator;
generating a cancelling audio signal, in one or more of the frequency ranges as selected by the operator, using the processor; and
emitting the generated cancelling audio signal, in the one or more frequency ranges as selected by the operator, using the speaker,
wherein at least portions of the cancelling audio signal comprise a predetermined phase shift relative to a phase of the detected incoming sound waves.

9. The method of claim 8, wherein the step of generating comprises adjusting a frequency of the cancelling audio signal to match a frequency of the detected incoming sound waves.

10. The method of claim 8, further comprising selecting a frequency range of the incoming sound waves to be detected in response to the one or more of the frequency ranges as selected by the operator and generating the cancelling audio signal in response to the selected frequency range of the incoming sound waves.

11. The method of claim 10, further comprising selecting the frequency range to include a frequency range of human voices.

12. The method of claim 10, further comprising providing a mechanical drive system for driving the wheels of the transport frame and presenting on the interface screen a frequency range of sound waves produced by the mechanical drive system.

13. The system of claim 1, further comprising a plurality of user selectable attenuation levels presented on the interface screen for each of the frequency ranges presented thereon, wherein the outgoing audio interference signal emitted over the speaker attenuates the portions of the incoming sound waves by an amount corresponding to the selected attenuation level.

14. The method of claim 8, further comprising:
presenting a plurality of variable attenuation levels, for each of the presented frequency ranges on the interface screen, for selection by an operator; and
emitting the generated cancelling audio signal, at the attenuation level selected by the operator, using the speaker.
